(19) United States
(12) Patent Application Publication
(10) Pub. No.: US 2022/0314017 A1
(43) Pub. Date: Oct. 6, 2022

(54) MAGNETIC FIELD EMITTER

(71) Applicant: Essence Security International (E.S.I) Ltd., Herzliya Pituach (IL)

(72) Inventors: Haim Amir, Herzlia (IL); Ohad Amir, Herzlia Pituach (IL); Doron Brosh, Herzlia (IL); Sergey Meron, Netanya (IL)

(21) Appl. No.: 17/619,901

(22) PCT Filed: Jun. 24, 2020
(86) PCT No.: PCT/IL2020/050703

§ 371 Date: Dec. 16, 2021

Related U.S. Application Data
(60) Provisional application No. 62/865,817, filed on Jun. 24, 2019.

(51) Int. Cl. A61N 2/12; A61N 2/06

(52) U.S. Cl. CPC A61N 2/12; A61N 2/06

(57) ABSTRACT

A magnetic field emitter (100) for emitting a magnetic field into an environment having a rotatable magnetic member (102) comprising one or more magnetic dipoles; a support (104) for the rotatable magnetic member (102), wherein the support (104) is configured to be placed in a support position on a surface of the environment such that when the support (104) is in the support position the one or more magnetic dipoles of the rotatable magnetic member (102) lie in a substantially horizontal plane; and a rotation mechanism (106) operable to rotate the rotatable magnetic member (102) relative to the support (104) about a rotation axis, such that when the support (104) is placed in the support position on the surface, the rotation axis is substantially orthogonal to said substantially horizontal plane such that the substantially horizontal plane remains substantially horizontal during the rotation.

### RELATED APPLICATION

This application claims the benefit of priority of U.S. Provisional Patent Application No. 62/865,817 filed on Jun. 24, 2019, the contents of which are incorporated herein by reference in their entirety.

### TECHNICAL FIELD

The present invention relates to a magnetic field emitter for emitting a magnetic field into an environment.

### BACKGROUND

Devices that use magnetic fields for therapeutic purposes are known. For example, U.S. Pat. No. 9,713,729 describes a device that has a magnet positioned relative to a head of a subject for providing therapy for example, for the treatment of coma and post-traumatic stress disorder. US 2005/0187423 describes a magnetic therapeutic device that is moved by hand and provides massage-like effects to a treatment area of a subject.

A disadvantage of the above devices is that they may only provide a local effect on a patient and therefore, in use, must be positioned close to a subject being treated. This may cause the device to be intrusive or awkward to use.

It is an aim of the invention to at least ameliorate one or more of the above or other shortcomings of the prior art and/or to provide a useful alternative.

### SUMMARY

A first aspect of the present invention provides a magnetic field emitter for emitting a magnetic field into an environment comprising:
a rotatable magnetic member comprising one or more magnetic dipoles;
a support for the rotatable magnetic member, wherein the support is configured to be placed in a support position on a surface of the environment such that when the support is in the support position the one or more magnetic dipoles of the rotatable magnetic member lie in a substantially horizontal plane; and
a rotation mechanism operable to rotate the rotatable magnetic member relative to the support about a rotation axis, such that when the support is placed in the support position on the surface, the rotation axis is substantially orthogonal to said substantially horizontal plane such that the substantially horizontal plane remains substantially horizontal during the rotation.

Thus, embodiments of the invention may provide a magnetic field emitter that is configured to be self-supporting in an operational position and which can emit a magnetic field into an environment along a substantially horizontal plane. As a result a user need not hold the emitter to provide a magnetic field to the user alone but can instead position the emitter to provide a magnetic field throughout an entire environment or region (e.g. a room) and many users may be able to experience the magnetic field from a single emitter.

The term “substantially” means within 40 degrees in some embodiments, within 30 degrees in some embodiments, within 20 degrees in some embodiments, within 10 degrees in some embodiments, and within 5 degrees in some embodiments.

The rotatable magnetic member may comprise a plurality of magnetic components, which may each contribute a magnetic dipole.

Said surface of the environment may be parallel to said substantially horizontal plane.

The one or more magnetic dipoles may be arranged to form a magnetic quadrupole. Each of the one or more magnetic dipoles of the magnetic quadrupole may lie in the substantially horizontal plane. The one or more magnetic dipoles may be arranged in a planar magnetic distribution.

At least one of the one or more magnetic dipoles may be provided by a permanent magnet. At least one of the one or more magnetic dipoles may be provided by an electromagnet.

The magnetic member may comprise a plurality of magnetic components, which may each contribute a magnetic dipole.

The rotation mechanism may comprise a drive arrangement operable to rotate the rotatable magnetic member, wherein the drive arrangement is electrically, magnetically and/or electro-magnetically powered. The rotation mechanism may comprise a motor, for example, a brushed DC motor, which is relatively cost effective.

The drive arrangement may be configured to rotate the rotatable magnetic member at a rotation frequency in a range 0.5 to 100 Hz, or between 1 Hz and 20 Hz, or between 6 and 7 Hz in some embodiments or at or about 10 Hz in other embodiments. In some embodiments the frequency is selectable, via a user and/or other input, from a set of predefined frequencies in any one of the above frequency ranges.

The emitter may further comprise a controller configured to control one or more operational parameters of the emitter thereby to control at least one of: turning the rotating of the rotatable magnetic member on and off; turning the emitter on and off; a frequency of rotation and/or a magnetic field strength. More specifically, the controller may be configured to control turning the rotating of the rotatable magnetic member on and off. Additionally or alternatively, the controller may be configured to control turning the emitter on and off. Additionally or alternatively, the controller may be configured to control a frequency of rotation. Additionally or alternatively, the controller may be configured to control a magnetic field strength.

Turning the emitter on and/or off may comprise moving the emitter into a higher and/or a lower power mode, for example, a standby and/or sleep mode.

The controller may be configured to control one or more operational parameters of the emitter thereby to turn off at least the rotating of the rotatable magnetic member when the support is not in the support position.

The emitter may comprise an electromagnet and controlling the one or more operational parameters of the emitter may comprise controlling the current provided to the electromagnetic and/or turning the electromagnet on and/or off.

The emitter as claimed any preceding claim comprising an orientation sensor for generating a measurement indicative of an orientation of the support and/or the rotatable magnetic member and a processor associated with said orientation sensor that is configured to process said measurement to determine whether the support is substantially in the support position and wherein said processor is further configured to instruct the controller to control one or more operational parameters of the emitter thereby to turn off at least the rotating of the rotatable magnetic member in response to determining that the emitter is not in the support position. The orientation sensor may be an accelerometer.

The controller may be configured to control one or more operational parameters of the emitter thereby to turn on at least the rotating of the rotatable magnetic member in response to detection of the presence of an object and/or an occurrence of an activity of an object in a region associated with the emitter. The controller may be configured to control one or more operational parameters of the emitter thereby to turn on at least the rotation of the rotatable magnetic member in response to identifying an object in a region associated with the emitter and detecting presence of an object and/or an occurrence of an activity of said identified object.

The controller may be configured to control one or more operational parameters of the emitter thereby to turn off at least the rotating of the rotatable magnetic member, in response to detection of an absence of an object and/or a period no occurrence of an activity of an object in a region associated with the emitter. For example, the time window may be 10 minutes, wherein if no activity is detected during a 10 minute period, it is determined that there is there an absence of the activity.

The controller may be configured to control one or more operational parameters of the emitter based on a measured distance between the emitter and an object in the environment.

The emitter may further comprise a processor associated with one or more sensors that is configured to receive sensor output from the one or more sensors and wherein said processor is further configured to process said sensor output to determine whether the sensor output is representative of a presence and/or an absence of an object and/or an occurrence of an activity of an object in a region associated with the emitter and wherein said processor is configured to instruct the controller to control one or more operational parameters in response to determining that the sensor output is representative of a presence and/or an absence of an object and/or of an occurrence of an activity of an object in a region associated with the emitter.

When determining that the sensor output is representative of a presence and/or an absence of an object and/or of occurrence of an activity of an object in a region associated with the emitter, the processor associated with the one or more sensors may be configured to perform a comparison process using said sensor output and at least one pre-determined threshold value.

The processor associated with the one or more sensors is further configured to generate a pre-defined time window in response to determining that said sensor output is representative of a presence of an object and/or of an occurrence of an activity of an object in the region associated with the emitter and wherein said processor is further configured to monitor sensor output for a further event that is representative of a presence of an object and/or of an occurrence an activity of an object in the region associated with the emitter, such that, in the absence of any further event in said time window, said processor instructs the controller to turn off at least the rotating of the rotatable magnetic member.

The detection of an absence of an object in a region associated with the emitter may additionally or alternatively be based on a detection of the object or an occurrence of an activity of an object in another region that does not overlap with said region associated with the emitter.

The emitter may comprise a processor. The processor may comprise the processor associated with the orientation sensor and/or the processor associated with the one or more sensors. The processor and the controller may be integrated into a single processor and/or processing chip. The emitter may further comprise a memory. The emitter may operate based on instructions stored on said memory. The emitter may operate based on instructions stored on an external memory. The single processor may further comprise the sensors and/or a communication module.

The emitter may further comprise the one or more sensors. The one or more sensors may comprise at least one of: a capacitive sensor, a photoelectric sensor, an inductive sensor, a radar sensor, a sonar sensor, a Passive Infrared (PIR) sensor, an acoustic sensor, an image sensor. For example, the image sensor may be configured for presence detection by image recognition or by motion detection via video motion detection.

The emitter may further comprise a communication module for receiving an external signal from a remote device, wherein the external signal is representative of a control signal and/or a presence and/or an absence of an object and/or an occurrence of an activity of an object in a region of the environment and wherein said controller is configured to control one or more operational parameters of the emitter in response to receiving said external signal.

The support may comprise a body having a dimension such that when in the support position the rotatable magnetic member is provided at a pre-determined distance from the surface. The pre-determined distance may be within a pre-determined range such that a user can adjust the pre-determined distance within the pre-determined range.

The support may be configured to extend substantially vertically when in the support position such that the support extends substantially orthogonal to the surface.

The support may be rotatably coupled to the magnetic member at a first position between the magnetic member and the surface of the environment and at a second position that is on an opposite side of the magnetic member to the first position. For example, this may ensure stability of the emitter during rotation, with the support causing minimal interference or blocking of the magnetic field.

The rotatable magnetic member may comprise a circular magnet that is substantially flat in a plane parallel to the rotational axis.

The emitter may be such that in the substantially horizontal plane, the rotatable magnetic member is substantially magnetically un-shielded.

The support may comprise a void in which the magnetic member is provided. The support may be such that the emitter is substantially open around the magnetic member.

The support may comprise a base for contacting the surface of the environment, and wherein the magnetic element has a first width and the base has a second width and wherein the second width is greater than the first width for stability.

The base may comprise a contact surface shaped to contact the surface of the environment, wherein the contact surface has a rotational symmetry. The contact surface may have a circular boundary.

The environment may be an interior environment, for example, a room, an enclosed space or a region thereof.

The emitter may further comprise an internal and/or removable power source.

The rotatable magnetic member may comprise a magnetically inert housing that encloses one or more magnets.

The rotation of the magnetic member may produce, at substantially every angular position adjacent the emitter that is coplanar with the substantially horizontal plane, a periodic magnetic waveform, wherein the magnetic waveform at each angular position has a frequency correlated with a speed of the rotation.

In a second aspect of the invention there is provided a method for affecting a physiological or psychological state of an occupant of an environment comprising:
providing a magnetic field emitter comprising:
a rotatable magnetic member comprising one or more magnetic dipoles;
   a support for the rotatable magnetic member, wherein the support is configured to be placed in a support position on a surface of the environment such that when the support is in the support position the one or more magnetic dipoles of the rotatable magnetic member lie in a substantially horizontal plane; and
   a rotation mechanism operable to rotate the rotatable magnetic member relative to the support about a rotation axis, such that when the support is placed in the support position on the surface, the rotation axis is substantially orthogonal to said substantially horizontal plane such that the substantially horizontal plane remains substantially horizontal during the rotation; and wherein the method further comprises
operating the magnetic field emitter to emit a rotating magnetic field into the environment.

Features in one aspect may be applied as features in any other aspect, in any appropriate combination. For example, method features may be provided as device features or vice versa.

### BRIEF DESCRIPTION OF THE SEVERAL VIEWS OF THE DRAWINGS

Embodiments will now be described by way of example only, and with reference to the accompanying drawings, of which:

FIG. 1 is a schematic diagram of a magnetic field emitter, in accordance with a first embodiment of the invention;

FIGS. 2(a) to 2(d) are perspective drawings representing a magnetic field emitter having a magnetic member, in accordance with a first embodiment of the invention;

FIG. 3 shows a magnetic field of a dipole;

FIG. 4 shows a magnetic field of a quadrupole;

FIG. 5 shows possible orientations of a rotatable magnetic member in accordance with embodiments of the invention; and

FIG. 6 shows a cross sectional view of an exemplary magnetic member in in accordance with FIGS. 2(a) to 2(d).

### DETAILED DESCRIPTION

As used herein, except where the context requires otherwise, the terms “comprises”, “includes”, “has”, and grammatical variants of these terms, are not intended to be exhaustive. They are intended to allow for the possibility of further additives, components, integers or steps.

Efforts have been made to apply certain frequencies of magnetic fields to a subject, for example those frequencies that are described herein, with the aim of affecting physiological or psychological states of the subject, with different frequencies used for different desired physiological or psychological state changes. Such physiological or psychological states include, for example, a subject's mood, stress level and/or anxiety level, and therefore changes in physiological or psychological state may have corresponding changes in measurable subject parameters, for example, a change in heart rate, breathing rate or pulse rate.

Without being limited to such an application, embodiments of the present invention relate to a magnetic field emitter that emits a time varying magnetic field into an environment. However, in some embodiments the physical properties of the emitted magnetic field in the environment may be used for the application described above.

In contrast to known devices that use a magnetic field for such therapeutic intent, emitters provided in accordance with embodiments of the present invention exemplified herein do not rely on providing a local magnetic effect for applying to a single subject. Instead, emitters provided in accordance with such embodiments provide a magnetic field into and throughout at least a region of an environment so that a global effect may be achieved throughout an area in which one or more subjects may be present.

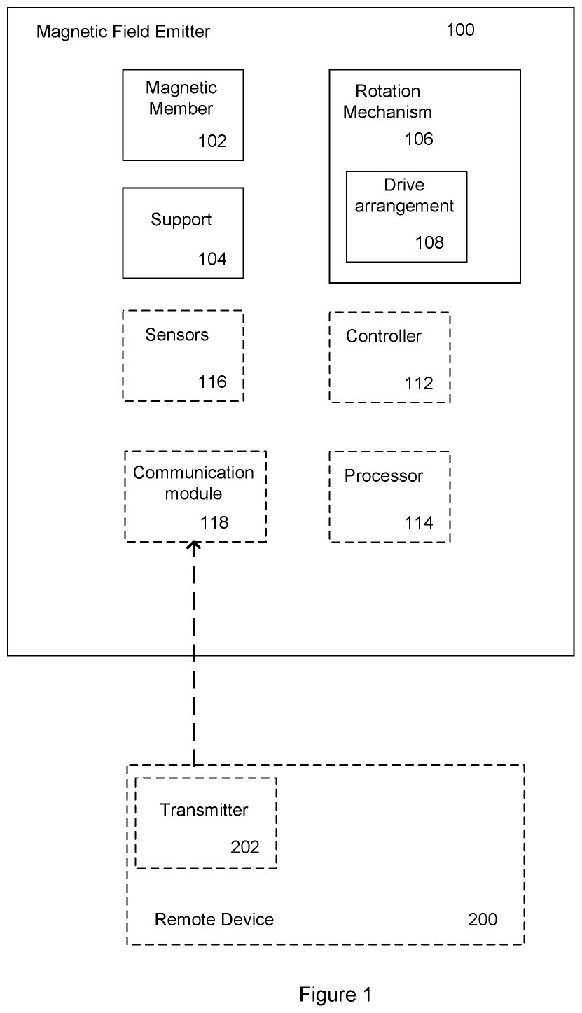

FIG. 1 shows a schematic diagram of a magnetic field emitter 100. For brevity the magnetic field emitter 100 may be simply referred to as an emitter 100. The emitter 100 includes a magnetic member 102 and a support 104. The magnetic member 102 is rotatable.

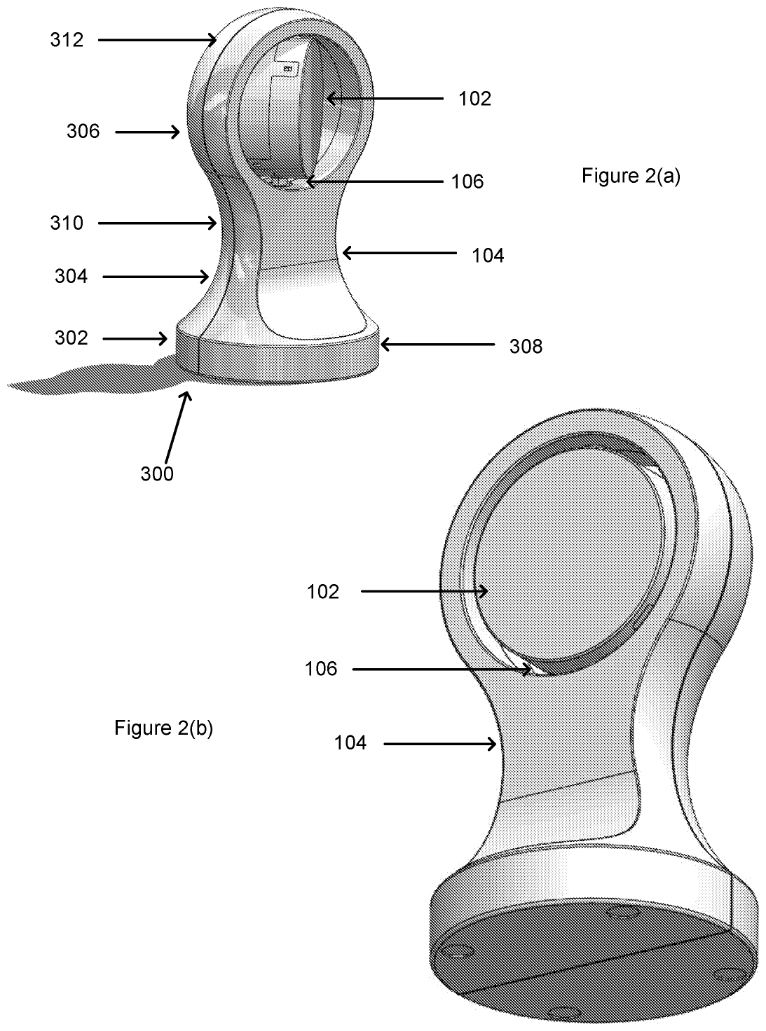

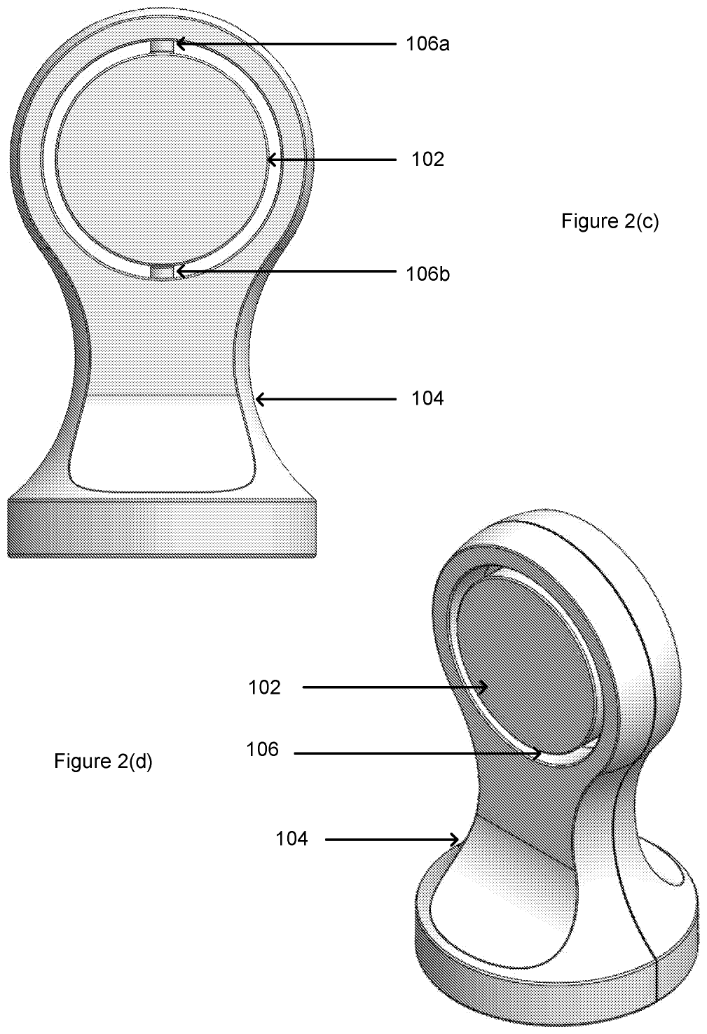

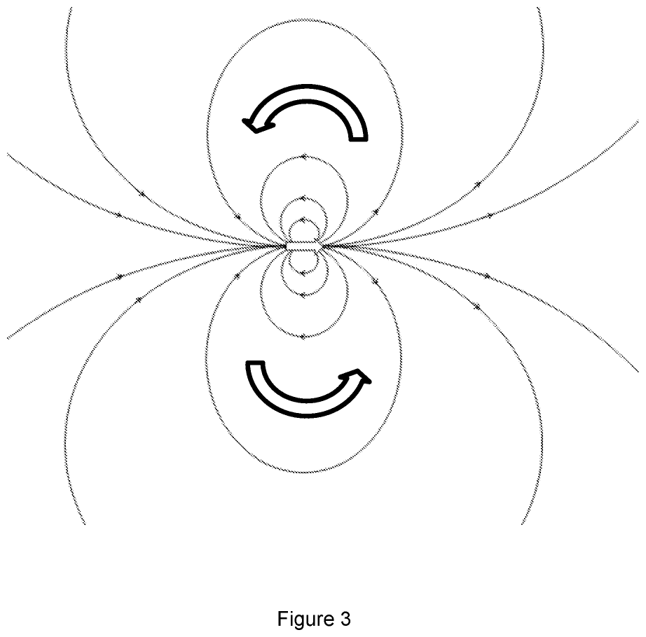

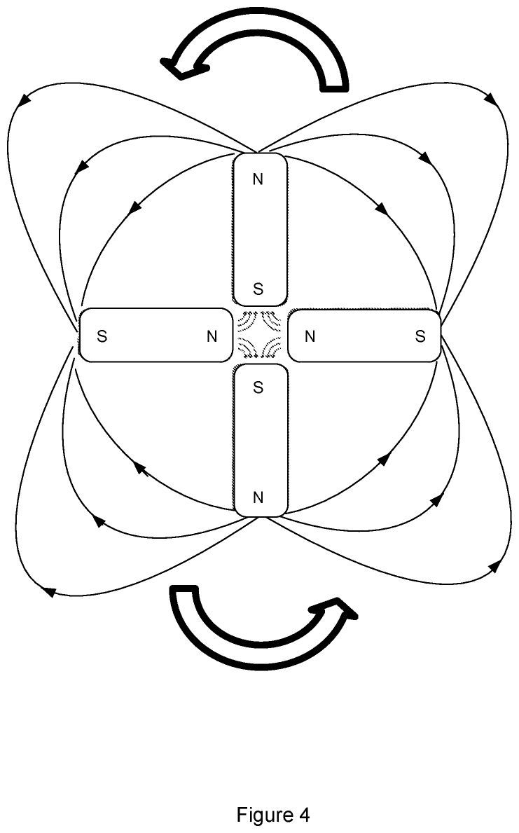

In the embodiments illustrated in FIGS. 2 and 3, the magnetic member 102 comprises a single magnetic element that provides a magnetic dipole. However, it will be understood that in other embodiments, for example as illustrated in FIG. 4, the magnetic member may include a plurality of magnetic components, which may each contribute a magnetic dipole.

The support 104 is configured to be placed in a support position on a surface of the environment. When the support 104 is placed in the support position the magnetic dipole of the rotatable magnetic member 102 lies in a substantially horizontal plane. In the present embodiment, the support position corresponds to the support 104 being placed on the surface such that the support 104 sits upright, substantially parallel to a vertical axis and orthogonal to the horizontal plane.

The emitter 100 also has a rotation mechanism 106 which is operable to rotate the magnetic member 102 about a rotation axis. The rotation mechanism 106 comprises rotatable couplings between the support 104 and the magnetic member 102. The orientation of the rotation axis is dependent on the positions and/or orientation of the support 104. In particular, when the support 104 is in the support position, the rotation axis is substantially orthogonal to the substantially horizontal plane in which the magnetic dipole of the rotatable magnetic member 102 lies and rotates. The horizontal plane may also be referred to as the plane of rotation. The rotation mechanism 106 maintains the substantially horizontal plane substantially horizontal as it is rotated. The rotation axis may also be described as parallel to a normal of the horizontal plane. The rotation axis may also be described as substantially vertical.

The rotation mechanism 106 has a drive arrangement 108 which is operable to rotate the rotatable magnetic member 102. In the present embodiment, the rotation mechanism 106 has a brushed DC motor. The drive arrangement is configured to rotate the rotatable magnetic member 102 at a rotation frequency. For example, the rotation frequency is in a range 0.5 to 100 Hz, or between 1 Hz and 20 Hz. In some embodiments the rotation frequency is more specifically in a range between 6 and 7 Hz, and in other embodiments at or about 10 Hz. The magnetic waveform generated at by the emitter 100 has a frequency correlated with the speed of rotation.

The drive arrangement may be configured to rotate the rotatable magnetic member at a rotation frequency in a range 0.5 to 100 Hz, or between 1 Hz and 20 Hz, or between 6 and 7 Hz in some embodiments or at or about 10 Hz in other embodiments.

The emitter 100 also has a controller 112 which is communicatively coupled to the rotation mechanism 106. In the present embodiment, the controller 112 is configured to control operational parameters of the emitter 100 thereby to perform each of the following: turning the rotation of the rotatable magnetic member on and off, turning the emitter on and off, changing a frequency of rotation and, in embodiments having an electromagnet, selecting a magnetic field strength. Turning the emitter off is herein intended to include turning the emitter to a sleep/dormant mode. In some embodiments, the controller 112 may be configured to perform any one or more of these tasks.

FIGS. 2(a) to 2(d) shows four perspective views of the magnetic field emitter 100, in accordance with an exemplary embodiment. FIGS. 2(a) to 2(d) show the rotatable magnetic member 102, support 104 and rotation mechanism 106.

As depicted in FIGS. 2(a) to 2(d), the support 104 has a base portion 302, a neck portion 304 and a head portion 306. The base portion 302 is shaped to contact the surface, which is depicted in FIG. 2(a) as surface 300. The neck portion 304 has a curved profile. The support 104 also has a front surface 308 and a back surface 310 and a curved side surface 312 provided as part of the neck portion 304 and head portion 306. The curved side surface 312 extends between the front surface 308 and back surface 310 such that the side surface 312 also provides an upper surface of the support 104.

FIGS. 2(b) to 2(d) show the emitter 100 in a particular configuration in which the rotatable magnetic member 102 lies in a flush orientation. Optionally, in some embodiments the magnetic member may have a specific rest orientation, and such embodiments such a rest orientation may correspond to the flush orientation. FIG. 2(a) shows the emitter 100 in a first rotated configuration in which the magnetic member 102 has been rotated through a first angle about the rotation axis. It will be understood that, although FIG. 2(a) shows only one rotated configuration, the magnetic member 102 is operable to be rotated into and through a continuum of rotated configurations where each rotated configuration corresponds to an angle of rotation about the rotation axis. As such the rotated configurations may be characterised by the angle of rotation. The magnetic member and/or each dipole of the magnetic member 102 lie substantially in the horizontal plane for all angles of rotation of the magnetic member. The dipole of the magnetic member 102 may be represented by North and South poles or a magnetic dipole vector and the rotation is such that the north and south poles or magnetic dipole vector remains in the horizontal plane during rotation.

In the illustrated embodiment, the head portion 306 has a circular void in which the rotatable magnetic member 102 is provided. The circular void is formed such that the head portion 306 provides a ring-portion characterised by a radius and a depth. The head portion 306 has parallel front and back ring surfaces joined together by the curved surface. The parallel front and back ring surfaces form part of the front surface 308 and back surface 310. The curved surface of the ring-portion extends between the parallel front and back ring surfaces and has the same size as the depth of the ring-portion. The curved surface of the ring-portion forms part of curved side surface 312. The configuration of the frame depicted in FIG. 2 may reduce interference and/or provide a substantially un-shielded space through which the magnetic field can be emitted from the rotatable magnetic member 102. By providing a ring-portion for the head portion 312, the support 104 is substantially open about the magnetic member 102 so that the magnetic field may be emitted in substantially all directions into the environment.

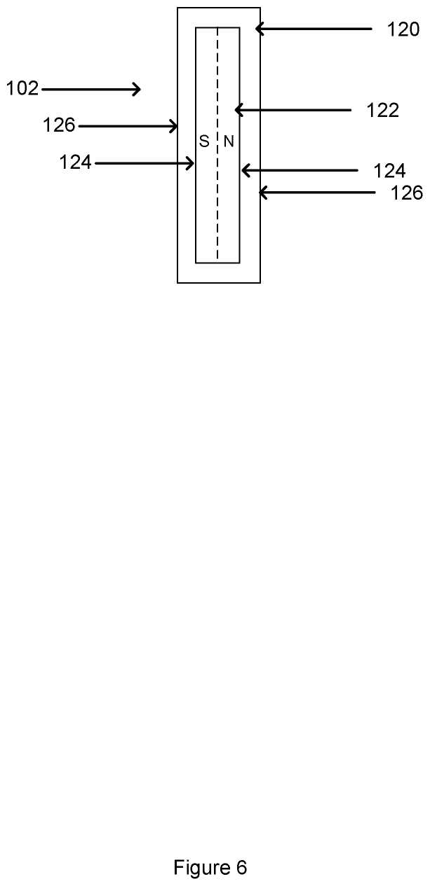

FIG. 6 shows a cross sectional view of an example of magnetic member 102 in accordance with FIG. 2. In this example, the rotatable magnetic member 102 has a housing 120 that encloses within it a right circular cylinder shaped magnet 122 that has a cylindrical North Pole and cylindrical South Pole. The housing 120 is in some embodiments magnetically inert. For example it may be composed of a magnetic permeability that is orders or magnitude less than conductive metals. For example the housing 120 may comprise, consist of, or consist essentially of a glass or a polymer, which may more specifically be a plastic polymer in some embodiments. The housing has cylindrical disk-like shape having a first circular plane surface and a second, parallel, circular plane surface and a curved side surface. The housing of rotatable magnetic member 102 shapes the magnetic member 102 as a right circular cylinder. The rotatable magnetic member 102 may be defined by the length of the curved side surface which corresponds to the distance between the first circular plane surface and the second circular plane surface and by the radius, or diameter, of the first and second circular plane surfaces. In the present embodiment, the first circular plane surface corresponds to a first pole of a magnetic dipole and the second circular plane surface corresponds to a second pole of the magnetic dipole.

The magnet 122 is held by the housing in a position that maintains a predefined minimum distance, Xmin, between each outer wall 124 of each pole of the magnetic 122 and the adjacent outer wall surface 126 of the plastic housing 120. The magnet 122 may have a relatively high magnetic pulling force, for example 64 kg in one example. Such a magnetic pulling force may pose a safety threat to a user, if the user is wearing a metallic object and accidentally being the object in close vicinity to the magnet 122 or if a finger ends up placed between the magnet 122 and another piece of metal. To reduce or remove this threat the minimum distance Xmin is selected to reduce the magnetic pulling force at the surface of the magnetic member 102 to a predefined acceptable maximum. The acceptable maximum may for example be 10 kg for some applications or a different value for other applications. In any case, the minimum distance may be selected to be not less than 4 mm in some embodiments for a 64 kg magnetic pulling force and also for other strength magnets.

The ring-portion of the head portion 306 has a depth that is substantially equal to the length of the magnetic member 102 to enable a rest configuration in which the first and second circular plane surfaces of the magnetic member 102 lies flush with the front and back surfaces of the ring-portion. The first and second circular plane surfaces of the magnetic element are substantially flat. The emitter 100 may optionally be configured into such a rest configuration by the motor in some embodiments or at least by manual manipulation in other embodiments.

As shown in FIG. 2(c) the magnetic member 102 is rotatably coupled to the support 104 via a first coupling 106a and a second coupling 106b. The second coupling 106b, and, in some embodiments the first couplings 106a, may be considered to form part of the rotation mechanism 106. The first and second couplings are provided along an axis that is parallel to the longitudinal axis of the support 104, which, when the support 104 is provided in the support position is substantially vertical. The couplings 106a, 106b are provided between the inner surface of the ring-portion of the head portion 306 and the side surface of the magnetic member 102. In particular, the couplings 106a, 106b are provided at opposing locations on the inner surface of the ring-portion. By providing couplings at opposing locations, the support 104 may provide enhanced stability for the magnetic member 102 and/or cause minimal interference to the emitted magnetic field.

As shown in FIG. 2(d) the base portion 302 has a circular shape with a diameter. The diameter of the base portion 302 is sized to be larger than the largest lateral (horizontal) dimension, the width, of the head portion 306 and magnetic member 102, and to be larger than the diameter of the ring-portion of the head portion 306. The base portion 302 is circular and thus has a rotational symmetry. By providing the base portion 302 that is wider that the head portion 306, the stability of the emitter 100 may be improved.

In use, the emitter 100 is provided on a surface in an environment, for example, a surface of a table. In further detail, a user manually handles the emitter 100 thereby to provide the support 104 in the support position so that the support 104 is substantially upright. When in the support position, the magnetic dipole of the magnetic member 102 is provided in a substantially horizontal plane. When the support 104 is in the support position, the controller 112 then provides a control signal to the drive arrangement 108 to start rotation of the rotatable magnetic member 102 about the rotation axis. The rotatable magnetic member 102 is then driven to rotate continuously, or for a controlled amount of time, at a specific rotation frequency, which may be a frequency selected from a set predefined rotation frequencies. The rotation of the rotatable magnetic member 102 occurs such that the dipole of the magnetic member 102 rotates in a substantially horizontal plane that is parallel to the surface. The rotation of the dipole occurs at the rotation frequency.

By rotating the dipole(s) at the rotation frequency, a time varying magnetic field is emitted into the environment, whereby the rotation of the magnetic member 102 produces at a plurality of positions in the environment a periodic magnetic waveform. The magnetic waveform at the plurality of positions has a frequency that is correlated with the speed of rotation.

The magnetic field emitter 100 is configured to emit a magnetic field into a region of the environment. It will be understood that the surface of the environment on which the support is placed may not be in this region. It may be that there is negligible magnetic effect on the place on the surface at which it the support is mounted.

The environment may be an interior environment or an exterior environment. Thus, the environment may be a room or a building such as a workplace, school, public building or a private home environment, or the environment may be an outdoor environment, for example, a garden or park.

FIG. 3 shows a magnetic field produced by a single magnetic dipole. In the above described embodiment, a magnetic member 102 that provides a single magnetic dipole is described. It will be understood that in other embodiments, other magnetic pole distributions may be provided. For example, FIG. 4 shows a magnetic field produced by four dipoles arranged to form a magnetic quadrupole. FIGS. 3 and 4 show magnetic field lines in the plane of rotation. FIGS. 3 and 4 also indicate a direction of rotation that is in accordance with an embodiment of the present invention.

A dipole that lies in a substantially horizontal plane is described above. In addition, the rotation axis is described as substantially orthogonal to said substantially horizontal plane. In the present embodiment, the dipole is considered to be lying in the horizontal plane when in a pre-defined angular range about a horizontal axis. Likewise, the rotation axis is considered orthogonal to the horizontal plane when in a pre-defined angular range about a vertical axis.

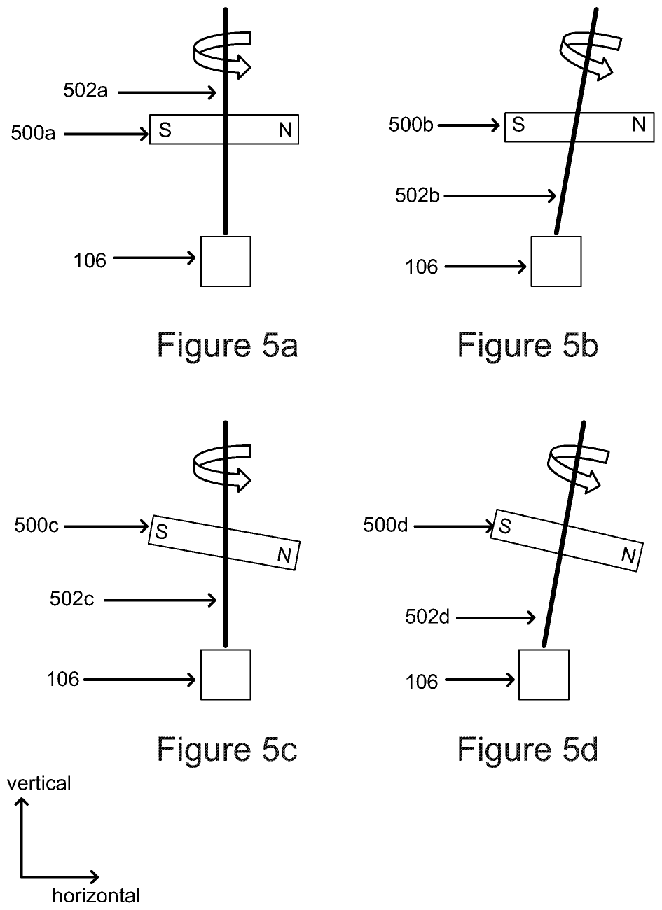

In the some embodiments, as illustrated in FIG. 5, the pre-defined range provides a tolerance of up to 10 degrees for angular measurements. However in other embodiments larger or smaller tolerances may be permitted. FIG. 5 shows example orientations of the dipole of the magnetic member 102 and rotation axis. FIG. 5 also shows the rotation mechanism 106 of the emitter 100.

The directions illustrated in FIG. 5 are defined relative to vertical and horizontal axes. FIG. 5(a) shows a dipole 500a that is parallel to the horizontal axis. FIG. 5(a) also shows the rotation axis 502a about which dipole 500a rotates. FIG. 5(a) shows a rotation axis 502a that is provided at a 90 degree angle (i.e. orthogonal) to the horizontal plane in which dipole 500a lies and is parallel to the vertical axis.

FIG. 5(b) shows dipole 500b with the same horizontal orientation as shown in FIG. 5(a). In contrast to FIG. 5(a), rotation axis 502b is at a deflected angle to the vertical axis. However, in the present embodiment, the rotation axis 502b is considered to be substantially orthogonal to dipole 500b as the rotation axis 502b falls within the pre-defined angular range. In other words, the angle between rotation axis 502b and the plane in which dipole 500b lies is within the range 90±10 degrees.

FIG. 5(c) shows rotation axis 502c parallel to the vertical axis as shown in FIG. 5(a). In contrast to FIG. 5(a), dipole 500c lies at a deflected angle to the horizontal axis. However, dipole 500c is considered to be substantially in the horizontal plane as it is oriented within ±10 degrees from the horizontal axis. Furthermore, the rotation axis 502c is considered to be substantially orthogonal to the dipole 500c as the angle between the rotation axis 502c and the dipole 500c is in the range 90±10 degrees.

FIG. 5(d) shows both rotation axis 502d and dipole 500d at angles that are deflected from the vertical and horizontal axes. However, dipole 500d is considered to be substantially oriented in the horizontal plane as it is within ±10 degrees from the horizontal axis. Furthermore, rotation axis 502d is considered to be orthogonal to the horizontal plane as it is provided at an orientation such that the angle between the rotation axis 502d and the horizontal plane is 90 degrees.

As will be appreciated, in each of FIGS. 5(a) to 5(d) the magnetic dipole is maintained in a substantially horizontal plane during rotation of the magnetic member.

Returning to FIG. 1, further features of the emitter 100, in accordance with further embodiments are now described. In a further embodiment, the emitter 100 has one or more sensors 116, a processor 114 and a communication module 118. The processor 114 is in communication with the sensors 116 and the controller 112. In some embodiments, the emitter 100 has a removable battery source (not shown).

The processor 114 is configured to receive sensor output from the one or more sensors 116 and is further configured to process said sensor output to determine whether the sensor output is representative of a presence and/or an absence of an object and/or an occurrence and/or a period no occurrence of an activity of an object in a region associated with the emitter 100. In response to determining that the sensor output is representative of a presence and/or absence of an object, the processor 114 is configured to instruct the controller 112 to control one or more operational parameters in response to determining that the sensor output is representative of a presence and/or an absence of an object and/or an occurrence and/or a period of no occurrence of an activity of an object in a region associated with the emitter 100.

In some embodiments, the one or more sensors 116 include: an activity sensor and/or a presence sensor and/or an orientation sensor. In the following, further non-limiting example embodiments are described.

The controller 112 is configured to control one or more operational parameters of the emitter 100 thereby to turn on at least the rotating of the rotatable magnetic member 102 in response to detection of the presence of an object and/or an occurrence of activity of an object in a region associated with the emitter 100. The controller 112 is further configured to control one or more operational parameters of the emitter 100 thereby to turn off at least the rotating of the rotatable magnetic member 102, in response to detection of an absence of an object and/or a period no occurrence of an activity of an object in a region associated with the emitter 100. As described in the following, detection of the absence and/or the presence of an object or of an occurrence or absence an activity of an object may be performed using a combination of at least one sensor and at least one processor for processing sensor output.

In accordance with a first further embodiment, the one or more sensors 116 include a sensor for sensing presence of an object within a certain region. In some embodiments sensor the region may be centred the about the emitter. The presence sensor may for example be proximity sensor, which is configured to produce sensor output in response to sensing a presence of an object a predefined distance from the sensor and/or from the emitter. The sensor output is sent from the proximity sensor to processor 114 and processed thereby to determine a proximity-correlated measurement. The processor 114 then performs a comparison process on the determined proximity correlated measurement. If the value of the determined proximity correlated measurement is determined to be less than a pre-determined threshold value then the processor 114 instructs the controller 112 to control the rotation mechanism 106 thereby to start one or more operations of the emitter 100. If the value of the determined proximity correlated measurement is determined to be greater than a pre-determined threshold value then the processor 114 instructs the controller 112 to control the rotation mechanism 106 thereby to stop one or more operations of the emitter 100.

Alternatively, the presence sensor may be sensor for identifying and positioning an object. The sensor may for example be a radar or sonar sensor. The processor 114 determines whether an object having certain characteristics is detected and/or detected within a region associated with the emitter. If such a detection occurs, then the processor 114 instructs the controller 112 to control the rotation mechanism 106 thereby to start one or more operations of the emitter 100. If a detected object is determined to have left the region, then the processor 114 instructs the controller 112 to control the rotation mechanism 106 thereby to stop the one or more operations of the emitter 100.

In accordance with a second further embodiment, the one or more sensors 116 include an activity sensor. The activity sensor is configured to produce sensor output in response to a certain activity of an object in a region associated with the emitter 100. The sensor output is transmitted from the activity sensor to processor 114 and processed thereby to determine if the sensor output corresponds to an occurrence of the activity. If the processor 114 determines that the activity has occurred then the processor 114 instructs the controller 112 to control the rotation mechanism 106 thereby to start one or more operations of the emitter 100.

The activity sensor is in some embodiments a motion sensor, wherein the activity is motion.

In other embodiments, activity sensor is an acoustic sensor (e.g. a microphone) wherein the activity is an action that produces an acoustic wave having one or more acoustic signatures that may be associated with an object of interest. The processor 114 may analyse the acoustic sensor output to determine, for example, whether a received sound may have been generated by a human. The acoustic signatures may for example one or more of: a sound classifiable as a human voice; a sound classifiable as a voice from a particular person; one or more predefined spoken words; a sound correlated with an action generally instigated by a person, e.g. clapping; footstep; closing of a cupboard door.

In accordance with these or other embodiments, the one or more sensors 116 include an activity sensor and/or presence sensor and the processor 114 may be configured to generate a pre-defined time window in response to determining that said sensor output is representative of an occurrence of an activity of an object of presence of an object in the region associated with the emitter 100. The processor 114 then monitors sensor output for a further event that is representative of a presence of an object and/or an occurrence of an activity of an object in the region associated with the emitter 100, such that, in the absence of any further event in said time window, the processor 114 instructs the controller to stop rotation of the rotatable magnetic member 102 of the emitter 100.

The controller 112 starting one or more operations of the emitter 100 may include turning the emitter 100 on, or to a higher power mode (e.g. from a sleep mode). In embodiments with an electromagnet, starting an operation may include providing a current to the electromagnet. In some embodiments, starting an operation may include changing the rotation frequency to a non-zero value, e.g. by starting a commanded rotation of the magnetic member.

The controller 112 stopping one or more operations of the emitter 100 may include turning the emitter off or to a lower power mode, for example, a sleep mode. In embodiments with an electromagnet, stopping an operation may include not providing a current to the electromagnet. In other embodiments, stopping an operation may include changing the rotation frequency to a zero value, e.g. by changing a commanded rotational speed, putting the magnetic member into a rest configuration, and/or by disabling the motor.

In a further embodiment, the one or more sensors 116 include an accelerometer for measurement of orientation. In this embodiment, the accelerometer is configured to generate a measurement of orientation of the support 104. The processor 114 is in communication with the accelerometer and is configured to process the measurement of orientation to determine whether the support 104 is in the support position. Determining that the support 104 is in the support position includes processing the measurement of orientation and comparing the value to pre-determined values.

In the present embodiment, the processor 114 determines that the value of measurement of orientation of the support 104 is outside a pre-determined range about an upright direction. The values of the pre-determined range are, for example, one to five degrees deflection from the vertical. However, it will be understood that different values can be used.

In response to determining that the support 104 is not in a tolerated range about the support position, the processor 114 is configured to instruct the controller 112 to stop one or more operations of the emitter 100.

For the above described embodiments, it will be understood that, instructing the controller 112 to stop and/or start one or more operations of the emitter 100 may correspond to providing a plurality of control signals to one or more components of the emitter 100. As a non-limiting example, control signals corresponding to an instruction to stop the emitter 100 may include the combination of: a stop rotation signal to the rotation mechanism 106 and/or, for example, a signal to stop providing a current to an electromagnet.

In a further embodiment, the emitter 100 has a communication module 118 that is configured to receive signals sent by a transmitter 202 of a remote device 200. Processor 114 is configured to process the received signals. In a first example embodiment, the remote device 200 is configured to transmit a signal representative of a control instruction from the remote device 200 and the communication module 118 is configured to receive said signal. Remote operation can be achieved by operating the remote device 200.

In the above described embodiments, the one or more sensors 116 are described as part of the emitter 100. It will be understood that, in accordance with further embodiments, the one or more sensors 116 are provided separately to the emitter 100 and the emitter 100 is configured to receive, via the communication module 118, signals representative of a presence and/or absence and/or a presence and/or absence of an occurrence of an activity in a region associated with the emitter 100. In some embodiments, the signals may also be representative of, for example, an orientation of a part of the emitter 100. In some embodiments, the signals received carry sensor data that is to be processed by processor 114 to determine a detection signal. In some embodiments, the signals are detection signals based on an output of a remote sensor such as any activity or presence sensor described herein. Such an output may be received directly from the remote sensor, but in some embodiments is received from a control panel that is in communication with the remote sensor. In these embodiments, the processor 114 can perform a comparison process directly on the detection signal.

In the above described embodiments, activity and presence sensors were described. It will be understood that different types of activity and presence sensors may be suitable for use with emitter 100.

For example, presence sensors may be one or more of: a capacitive sensor, photoelectric sensor, an inductive sensor, a radar (microwave or radiowave) sensor and/or a sonar sensor. In some embodiments, proximity detection may be based on a pass or fail distance. In some embodiments, a measurement of distance is made for example, by radar or sonar. In other embodiments, some other threshold that is less precisely correlated with distance is used, for example, a measurement by a capacitive sensor.

Motion detection may, for example, be based on: a passive infrared (PIR) sensor, video motion detection, radar, for example, microwave or radiowave and/or sonar. However, in some embodiments, motion detection is more specifically based on at least a PIR sensor. It will be understood that any of the proximity detectors may also potentially be used as for motion detection based on a change of a measured proximity. Likewise position and tracking systems (e.g. using video, radar and/or sonar) may also be for motion detection. A further option for proximity and/or motion detection is Lidar.

As described above, orientation measurement may be provided by an accelerometer or any other gyroscope based sensor. In the above described embodiment, an accelerometer is provided on the support 104. It will be understood that the accelerometer may be provided on other parts of the emitter 100. For example, the accelerometer may provide a measurement of orientation of the rotatable magnetic element 102.

A skilled person will appreciate that variations of the described arrangements are possible without departing from the invention. Variations of the described arrangement are described in the following.

In some embodiments, the magnetic member 102 may be stopped when in the rest configuration described herein, for example, to allow for easier transportation. In the embodiments shown in FIG. 2, this is when the magnetic member 102 is flush with the ring of the head portion 306. In such embodiments, instructing the controller 112 to stop and/or start one or more operations of the emitter 100 may correspond to the rotation mechanism 106 rotating the rotatable member 102 thereby to reset the position of the rotatable magnetic member 102 to the rest configuration.

In some embodiments, the controller 112 is configured to control one or more operational parameters of the emitter 100 based on a measured distance between the emitter 100 and an object in the environment. In some embodiments, where the one or more sensors include, for example, an image sensor, the processor 114 is further configured to perform a step of identifying a type of object in the environment or determining that the object in the environment belongs to a particular class of objects. The class of objects may consist of, or comprise, humans. The image sensor may perform presence detection by image recognition; or by motion detection via video motion detection.

In the above described embodiments, the drive arrangement 108 is a brushed DC motor. However, it will be understood that, in other embodiments, the drive arrangement may be any suitable electrically, magnetically and/or electro-magnetically powered motor. In the case of a brushed DC motor or other motors that rely on closed loop control, the emitter 100 may include one or more position and/or magnetic feedback sensors. Magnetic feedback sensors may include one or more hall effect sensors and/or other magnetometers, and/or a magnetic rotary encoder. Other rotary encoders may alternatively be used for measuring position for feedback. Feedback from such magnetometers may for example be used to control motor speed, while feedback from a position encoder may be used to control motor speed and/or for relatively precisely controlling a stopping orientation of the magnetic member.

In the illustrated embodiment, the magnetic member 102 comprises a permanent magnet. A magnetized material may be used for the magnetic member 102. In other embodiments, electromagnets, for example, a solenoid, are used in the magnetic member 102, instead of and optionally in the pace of permanent magnet 122, to allow for control of magnetic field strength. In embodiments with an electromagnet, one or more operational parameters related to the electromagnet can be controlled to modify the generated magnetic field strength. For example, the current provided to the electromagnet may be modified thereby to modify the generated magnetic field strength.

Although the embodiment depicted FIG. 2 has a circular void, in other embodiments any shape of void or any suitable structure that provides an un-shielded and/or substantially open frame for the magnetic element may be used. While in the above embodiment, a support 104 having a ring-shaped head portion 306 is described, it will be understood that alternative shapes and/or structures may be provided for supporting the magnetic member 102, in accordance with other embodiments. For example, the support 104 may have a frame that is substantially open allowing the magnetic field to be emitted.

Although shown in FIG. 1 as two separate components, it will be understood that the controller 112 and the processor 114 may be provided as a single integrated chip. The processor 114 and the controller 112 may be integrated into a single processor/processing chip (that may optionally operate based on instructions stored an internal or external memory), which additionally or alternatively may include the sensors 116 and/or the communication module 118.

Although not shown in FIG. 2, other components of the emitter 100, for example, sensors 116, controller 112, communication module 118, processor 114 and/or the removable battery source, are provided in the neck portion 304 and/or the base portion 302 of the emitter 100.

In the above embodiments, the emitter 100 has a memory (not shown) for storing instructions to be executed by the processor 114. The memory also stores threshold values and/or other values required by the processor 114.

In some embodiments, the emitter 100 has a removable and/or rechargeable battery source (not shown). In some embodiments, the emitter 100 uses a mains power source. Controlling operational parameters in response to control signals and/or detection of an object's presence and/or activity may have several advantages. It may allow for power saving advantages and/or less wear and tear on the emitter. Additionally or alternatively, it may reduce risk of damage to the emitter and/or objects that it may be incidentally touching when the emitter's use it not needed.

In the above described embodiments, emitter 100 is described as being provided in a horizontal flat support position. In such embodiments that horizontal flat support position may be elevated from a floor, for example as may be provided by a table top, to bring the horizontal plane of the magnetic member rotation closer an upper portion of an adjacent sitting or standing person. Emitters in accordance with other embodiments are provided to be mounted some other surface in an environment. In a further embodiment, the emitter has a support configured to be mounted to a wall, floor or a ceiling. As will be appreciated, for such other mounting surfaces, the emitter may have a different shape to that illustrated in the drawings.

Whilst the foregoing description has described exemplary embodiments, it will be understood by those skilled in the art that many variations of the embodiments can be made within the scope of the present invention as defined by the claims. Moreover, features of one or more embodiments may be mixed and matched with features of one or more other embodiments.

1. A magnetic field emitter for emitting a magnetic field into an environment comprising:
a rotatable magnetic member comprising one or more magnetic dipoles;
a support for the rotatable magnetic member, wherein the support is configured to be placed in a support position on a surface of the environment such that when the support is in the support position the one or more magnetic dipoles of the rotatable magnetic member lie in a substantially horizontal plane; and
a rotation mechanism operable to rotate the rotatable magnetic member relative to the support about a rotation axis, such that when the support is placed in the support position on the surface, the rotation axis is substantially orthogonal to said substantially horizontal plane such that the substantially horizontal plane remains substantially horizontal during the rotation.

2. The emitter as claimed in claim 1, wherein said surface of the environment is parallel to said substantially horizontal plane.

3. The emitter as claimed in claim 1, wherein the one or more magnetic dipoles are arranged to form a magnetic quadrupole.

4. The emitter as claimed in claim 1, wherein at least one of the one or more magnetic dipoles is provided by a permanent magnet.

5. The emitter as claimed in claim 1, wherein at least one of the one or more magnetic dipoles is provided by an electromagnet.

6. The emitter as claimed in claim 1, wherein the rotation mechanism comprises a drive arrangement operable to rotate the rotatable magnetic member, wherein the drive arrangement is electrically, magnetically and/or electro-magnetically powered.

7. The emitter as claimed in claim 6, wherein the drive arrangement is configured to rotate the rotatable magnetic member at a rotation frequency in a range 0.5 to 100 Hz, or between 1 Hz and 20 Hz, or at or about 10 Hz, or between 6 and 7 Hz.

8. The emitter as claimed in claim 1, comprising a controller configured to control one or more operational parameters of the emitter thereby to control at least one of: turning the rotating of the rotatable magnetic member on and off; turning the emitter on and off; a frequency of rotation and/or a magnetic field strength.

9. The emitter as claimed in claim 8, wherein the controller is configured to control one or more operational parameters of the emitter thereby to turn off at least the rotating of the rotatable magnetic member when the support is not in the support position.

10. The emitter as claimed in claim 8, comprising an orientation sensor for generating a measurement indicative of an orientation of the support and/or the rotatable magnetic member, wherein the emitter further comprises a processor associated with said orientation sensor that is configured to process said measurement to determine whether the support is substantially in the support position and wherein said processor is further configured to instruct the controller to control one or more operational parameters of the emitter thereby to turn off at least the rotating of the rotatable magnetic member in response to determining that the emitter is not in the support position.

11. The emitter as claimed in claim 8, wherein the controller is configured to control one or more operational parameters of the emitter thereby to turn on at least the rotating of the rotatable magnetic member in response to detection of the presence of an object and/or an occurrence of activity of an object in a region associated with the emitter.

12. The emitter as claimed in claim 8, wherein the controller is configured to control one or more operational parameters of the emitter thereby to turn off at least the rotating of the rotatable magnetic member in response to detection of an absence of an object and/or a period of no occurrence an activity of an object during a defined time window, in a region associated with the emitter.

13. The emitter as claimed in claim 8, wherein the controller is configured to control one or more operational parameters of the emitter based on a measured distance between the emitter and an object in the environment.

14. The emitter as claimed in claim 8, wherein the emitter comprises a processor associated with one or more sensors, the processor being configured to receive sensor output from the one or more sensors and wherein said processor is further configured to process said sensor output to determine whether the sensor output is representative of a presence and/or an absence of an object and/or an occurrence and/or a period of no occurrence of an activity of an object in a region associated with the emitter and wherein said processor is configured to instruct the controller to control one or more operational parameters in response to determining that the sensor output is representative of a presence and/or an absence of an object and/or occurrence and/or a period of no occurrence of an activity of an object in a region associated with the emitter.

15. The emitter as claimed in claim 14, wherein when determining that the sensor output is representative of a presence and/or an absence of an object and/or an occurrence and/or a period of no occurrence of an activity of an object in a region associated with the emitter, the processor associated with the one or more sensors is configured to perform a comparison process using said sensor output and at least one pre-determined threshold value.

16. The emitter as claimed in claim 14, wherein the processor associated with the one or more sensors is configured to generate a pre-defined time window in response to determining that said sensor output is representative of a presence of an object and/or of an occurrence of an activity of an object in the region associated with the emitter and wherein the processor is further configured to monitor sensor output for a further event that is representative of a presence of an object and/or an occurrence of activity of an object in the region associated with the emitter, such that, in the absence of any further event in said time window, said processor instructs the controller to turn off at least the rotating of the rotatable magnetic member.

17. The emitter as claimed in claim 14, wherein the emitter further comprises the one or more sensors, comprising at least one of: a capacitive sensor, a photoelectric sensor, an inductive sensor, a radar sensor, a sonar sensor, a Passive Infrared (PIR) sensor, an acoustic sensor, an image sensor.

18. The emitter as claimed in claim 14, wherein sensor output comprises one or more of:
a motion sensor output and/or an acoustic sensor output, for determining an occurrence and/or a period of no occurrence of an activity of the object; and
a proximity sensor output for determining a presence and/or an absence of the object and/or sensor output for identifying and determining a position of an object for determining a presence and/or an absence of the object.

19. The emitter as claimed in claim 8, comprising a communication module for receiving an external signal from a remote device, wherein the external signal is representative of a control signal and/or a presence and/or an absence of an object and/or an occurrence of an activity of an object in a region of the environment and wherein said controller is configured to control one or more operational parameters of the emitter in response to receiving said external signal.

20. The emitter as claimed in claim 1, wherein the support comprises a body having a dimension such that when in the support position the rotatable magnetic member is provided at a pre-determined distance from the surface.

21. The emitter as claimed in claim 1, wherein the support is configured to extend substantially vertically when in the support position such that the support extends substantially orthogonal to the surface.

22. The emitter as claimed in claim 1, wherein the support is rotatably coupled to the magnetic member at a first position between the magnetic member and the surface of the environment and at a second position at an opposite side of the magnetic member to the first position.

23. The emitter as claimed in claim 1, wherein the magnetic member comprises a circular magnet that is substantially flat in a plane parallel to the rotational axis.

24. The emitter as claimed in claim 1, wherein, at least in the substantially horizontal plane, the magnetic member is substantially magnetically un-shielded.

25. The emitter as claimed in claim 1, wherein the support comprises a void in which the magnetic member is provided.

26. The emitter as claimed in claim 1, wherein the support comprises an open frame that holds the magnetic member.

27. The emitter as claimed in claim 1, wherein the support comprises a base for contacting the surface of the environment, and wherein the rotatable magnetic element has a first width and the base has a second width wherein the second width is greater than the first width.

28. The emitter as claimed in claim 27, wherein the base comprises a contact surface shaped to contact the surface of the environment, wherein the contact surface has a rotational symmetry.

29. The emitter as claimed in claim 28, wherein the environment is an interior environment, and
wherein the emitter further comprises an internal and/or removable power source.

30. (canceled)

31. The emitter as claimed in claim 1, wherein the rotatable magnetic member comprises a magnetically inert housing that encloses one or more magnets.

32. The emitter as claimed in claim 1, wherein the rotation of the rotatable magnetic member produces, at substantially every angular position adjacent the emitter that is coplanar with the substantially horizontal plane, a periodic magnetic waveform, wherein the magnetic waveform at each angular position has a frequency correlated with a speed of the rotation.

33. A method for affecting a physiological or psychological state of an occupant of an environment comprising:
providing a magnetic field emitter comprising:
a rotatable magnetic member comprising one or more magnetic dipoles;
a support for the rotatable magnetic member, wherein the support is configured to be placed in a support position on a surface of the environment such that when the support is in the support position the one or more magnetic dipoles of the rotatable magnetic member lie in a substantially horizontal plane; and
a rotation mechanism operable to provide rotation of the rotatable magnetic member relative to the support about a rotation axis, such that when the support is placed in the support position on the surface, the rotation axis is substantially orthogonal to said substantially horizontal plane such that the substantially horizontal plane remains substantially horizontal during the rotation; and wherein the method further comprises
operating the magnetic field emitter to emit a rotating magnetic field into the environment.
